(19) United States
(12) Patent Application Publication
(10) Pub. No.: US 2018/0125402 A1
(43) Pub. Date: May 10, 2018

(54) LANCET HOLDING DEVICE AND METHODS OF USE THEREOF

(71) Applicant: Christana Kmecz, Oakhurst, NJ (US)

(72) Inventor: Christana Kmecz, Oakhurst, NJ (US)

(21) Appl. No.: 15/347,916

(22) Filed: Nov. 10, 2016

(51) Int. Cl. A61B 5/15; A61B 5/151

(52) U.S. Cl. CPC A61B 5/150259; A61B 5/15186

(57) ABSTRACT

A blood lancing device and, more particularly, to an ergonomic blood lancet holding device and methods of use thereof that allow for single-handed operation, particularly by individuals with limited or no operational function in one arm or hand and by individuals with body tremors. Additional embodiments provide a universal lancet securing means for detachable holding of various proprietary lancet shapes, designs and formats that are presently or may become commercially available. Outer contours of embodiments are ergonomically shaped to readily accommodate the palmar surface of the hand and digits, thereby providing the user with easy-to-grasp functionality, without excess stretching or discomfort of the hand, digits, wrist or forearm. Embodiments may be symmetrical in design for universal use by either the right or left hand or specifically designed for use only by the right hand or the left hand.

### FIELD OF THE INVENTION

The present invention relates generally to blood lancing devices and, more particularly, to an ergonomic blood lancet holding device that allows for single-handed operation.

### BACKGROUND

Some diseases, such as diabetes, require that an individual test their blood on a daily (or more) basis. Such testing requires that a small sample of the individual's blood be obtained. Generally, this is accomplished by piercing or puncturing the skin to draw blood in an amount sufficient for testing. A skin piercing or puncturing device—a small medical implement used for capillary blood sampling—known as a blood lancet or lancet, is generally used for that task and there is a variety of commercially available lancet holding devices or lancet devices that utilize a variety of lancets, many proprietary in design and function for specific use with a device. Lancets may be used without a lancet device or holding device, in which case obtaining the blood sample requires that an individual lance the skin, typically the skin on the tip of a finger (digit) by holding a lancet in one hand and pressing it onto the fingertip on the other hand. As used herein, the term “digit” refers to any of the fingers, including the thumb, of either hand that may be suitable for lancing to obtain a blood sample for testing.

Lancets are generally comprised of a sharp, pointed piece of surgical steel (either a double-edged blade or needle) and are usually encased in a plastic body for holding or securing with a lancet device or by holding with a hand. The sharp, pointed end of the lancet is used to puncture the skin on one's finger (or other body part) to draw the blood sample. Alternatively, the lancet sharp end and body may be comprised from the same, single piece of surgical steel or other appropriate metal. The sharp, pointed needle portion of the lancet that punctures the skin is available in different gauges, which refer to the width of the surgical steel point. Lancet gauges generally range from about 20 to about 34 and the higher the gauge, the smaller the perforation the lancet makes. For example, a 23-gauge lancet creates a larger puncture wound in the skin than does a 30-gauge lancet. FIG. 1 depicts eight (8) commercially available blood sampling lancets 20, each of which is comprised of distal end 20a comprising a sharp, pointed needle element N and a proximal end 20b comprising a handle or body portion B which may be manually held by a user or secured within a lancet device or lancet holding device. Lancets are often sold in bulk packages and are generally disposable, that is, the lancet is disposed in a safe, secure manner after a single use.

As used herein, the term “lancet” 20 shall therefor refer to and mean a small surgical instrument or implement, comprising a distal end 20a that is comprised of a short, sharp-pointed, double-edged or needle-like blade element N (for making punctures and small incisions in a person's skin) and a proximal end 20b that is comprised of a wider body or handle portion B for manually grasping or holding or securing by or within a lancet holding device, such as the present invention. The wider body portion B is usually comprised of an injection molded thermoplastic, for example Polyamide (Nylon), ABS, Acetal, Polycarbonate (PC), Polyethylene (PE), Polypropylene (PP), or Polyvinyl chloride (PVC), which may further be blended or glass reinforced, though any other suitable material, such as a lightweight plastic or metal that is capable of being machined. Alternatively, the lancet body portion B may be comprised of the same surgical steel comprising the blade/needle element N or any other appropriate metal alloy as is generally known in the art. The body/handle portion B of the lancet 20 may further define one or more holes, features and elements that aid in coupling the lancet 20 to a specific, corresponding lancet device or lancet holding device, particularly proprietary commercial devices specifically designed for accepting, securing and using a particular lancet design (refer to FIG. 1 for such detail as to such holes, features and elements of various proprietary, commercial lancets 20).

While some users simply use a lancet alone to stick their fingers, many individuals prefer to use a lancing device. Lancing devices are commonly known in the art and may comprise, in some instances, a spring or other mechanically activated kinetic source to drive the lancet into the skin and retract it very quickly. Such devices typically also comprise a cocking, locking and triggering system by which the lancet puncturing action is actuated by a user. Such devices also may allow a user to change the depth of penetration depending on the thickness of the skin and calluses and the sensitivity of the fingertips.

Existing lancing devices, however, typically require two-handed operation and can be dangerous. Two-handed operation may require that a user hold the lancing device in one hand (the holding hand) and place the tip of a digit of the other hand (the target hand) against the puncturing portion of the lancet device, while the holding hand then activates or triggers the lancing device, whereby the lancet secured within is mechanically activated and the needle portion N thereof driven into the skin of the digit of the target hand. Furthermore, the activation of lancing devices is typically done in at least two steps: cocking (and locking) the device and triggering (actuating) the device to drive the lancet into the skin. This is inconvenient to many users as the user does not have the use of another hand while operating the lancing device, i.e., the process requires the use of two hands. Additionally, the two-step process of activating a lancing device causes safety issues because once cocked, a user may inadvertently fire the lancing device. By inadvertently firing a lancing device, one may unintentionally pierce one's or another's skin causing pain and discomfort or the transmittal of diseases.

Users with medical conditions such as diabetes or elderly users may have limited use of one arm or hand. In such cases, it may be difficult, impossible or simply not safe for such an individual to use or attempt to use a lancet device that requires two-handed operation. Rather than use a lancet device (which require to functional hands and/or arms for use), individuals with the use of only one hand or arm have been known to delicately balance a lancet 20 by placing the proximal end portion 20b on a flat, stable surface, such as, for example, on a table—with the distal end portion 20a comprising the needle element N pointing upward—and then quickly attempt to puncture the skin on the tip of a digit to obtain a blood sample. In such cases, the lancet may fall over before a puncture wound is inflicted, or worse, a larger and more painful wound may result from the slippage of an unsecure, unstable lancet. Other similarly situated individuals have also resorted to placing the proximal end 20b or body portion B of a lancet 20 between their knees or in other physically awkward and/or unsafe orientation in an attempt to securely hold the lancet while the one working arm is manipulated to obtain a blood sample by sticking a digit of the associated hand on the needle N of the distal end 20a of the lancet 20. The safety issues arising from such uses of blood lancets are self-evident and using lancets in such fashion should be avoided.

Individuals suffering from tremors (shaking), a condition or symptom typically associated with old age (e.g., essential tremor), Parkinson's Disease, multiple sclerosis (MS), and other diseases, conditions, disorders, etc. also encounter difficulty when using lancets. Manual use, i.e., without a lancet device or lancet holding device, may result in failure to puncture or adequately puncture the skin, or the puncture may not be a “clean” piercing of the skin, thereby cause more trauma to the skin and pain to the user than is necessary to obtain the blood sample. In addition, several attempts to obtain an appropriate puncture that results in sufficient blood draw may be required. Attempting to use a mechanically driven lancet device may also result in premature triggering or actuation of the device, again requiring multiple attempts to obtain a blood sample. Any factor that prolongs the process of obtaining a lancet-induced blood sample—particularly, if multiple puncture attempts must be performed—or that creates unsafe blood drawing practices, necessarily results in an unpleasant experience for the individual. This is especially true if multiple samples are required within a single day, thereby producing anxiety and aversion to the blood sampling procedure in the individual.

What is needed is a simple, efficient, ergonomic and economic lancet holding device that firmly and safely holds a blood lancet for use (puncturing or piercing the skin to obtain a blood sample for testing) that does not involve complex mechanical functionality (e.g., “lock, cock and trigger”) typically found in many lancet devices that is susceptible to wear and tear, breakage and malfunction, such as that which may occur to the device, for example, if it is dropped onto a hard floor or surface.

What is further needed is an easy-to-use, ergonomic lancet holding device that meets the needs of individuals with limited or no operational function or use in one arm or hand, i.e., an easy-to-use, ergonomic lancet holding device that allows for simple, easy single-handed operation.

What is further needed is an easy-to-use, simple and safe, ergonomic lancet holding device that allows for stable and safe use by individuals suffering from any tremor condition or symptom, for instance, such conditions or symptoms typically associated with older age (e.g., essential tremor), Parkinson's Disease, multiple sclerosis (MS), and other diseases, conditions, disorders, etc.

What is further needed is a lancet holding device that provides universal lancet adaptability, i.e., that works with and functionally operates in a safe, stable fashion with the myriad of lancets commercially available and is not dependent on any particular or proprietary lancet design, shape, size or format.

It would be desirable to have a lancing holding device and a method for using that device that addresses these issues.

### SUMMARY OF THE INVENTION

The present invention relates generally to blood lancing devices and, more particularly, to an ergonomic blood lancet holding device that allows for single-handed operation of the device.

More specifically, the present invention is directed to a simple, efficient, ergonomic and economic lancet holding device that firmly and safely holds a blood lancet for use (puncturing or piercing the skin to obtain a blood sample for testing) that does not involve mechanical functionality (e.g., “lock, cock and trigger”) typically found in many lancet devices, in which cases, such functionality is susceptible to wear and tear, breakage, malfunction, etc. In addition, the present invention is further directed to an easy-to-use, simple and safe, ergonomic lancet holding device that meets the needs of individuals with limited or no operational function in one arm or hand, thereby allowing for single-handed operation. The invention is also directed to an ergonomic lancet holding device that allows for stable and safe use by individuals suffering from any tremor condition or symptom, for instance, such conditions or symptoms typically associated with older age (e.g., essential tremor conditions), Parkinson's Disease, multiple sclerosis (MS), and other diseases, conditions, disorders, etc. Lastly, the present invention is further directed to a lancet holding device that provides universal lancet adaptability, i.e., the lancet holding device works with and functionally operates in a safe, stable fashion with a myriad of lancets that are presently or may become commercially available and the invention is not dependent on any particular or proprietary lancet design, shape, size or format.

### BRIEF DESCRIPTION OF THE DRAWINGS

FIG. 1 is perspective view of several proprietary designs or types of commercially available blood lancets.

FIG. 2 is perspective view of an embodiment of the invention.

FIG. 3 is perspective view of an embodiment of the invention and further depicting a user placing a lancet in the device.

FIG. 4 is a further perspective view of an embodiment of the invention in use, wherein a hand of a user is depicted gripping the device and ready to lance a digit.

FIG. 5 is a further perspective view of an embodiment of the invention in use (opposite from the perspective view of FIG. 4), wherein a hand of a user is depicted grasping the device and ready to lance a digit.

FIG. 6 is a cross section view of a portion of an embodiment of the invention.

FIG. 7A is a top view of a portion of an embodiment of the invention, in particular, a top view of a lancet receiving receptacle feature of an embodiment.

FIG. 7B is a top perspective view of a portion of an embodiment of the invention, in particular, a top perspective view of a lancet receiving receptacle feature of an embodiment.

FIG. 7C is a cross section view of a portion of an embodiment of the invention, in particular, a cross section view of a lancet receiving receptacle feature of an embodiment.

FIG. 7D is a top perspective view of a portion of an embodiment of the invention, in particular, a top perspective view of a lancet receiving receptacle of an embodiment.

FIG. 8 is a simple perspective view of an embodiment of the invention.

FIG. 9 is a simple side view of a further embodiment of the invention.

FIG. 10 is a perspective view of a further embodiment of the invention for use with a right hand.

FIG. 11 is a top view of a further embodiment of the invention.

FIG. 12 is a perspective view of a further embodiment of the invention for use with a right hand.

FIG. 13 is a perspective view of a further embodiment of the invention for use with a left hand.

### DRAWING REFERENCES

Various embodiments of the invention are disclosed in the drawings.

For ease of reference between this disclosure and among the attached illustrations, identical reference numbers are used for various features, limitations, portions, elements, etc. of the various embodiments. The following are the various reference numbers and the associated elements and features of the embodiments and attachments as shown in the attached illustrations:

### 100 Lancet Holding Device (Embodiment) References

4 Bottom portion
4a Base element
5 Bottom surface
6 Outer contour surface of main body
8 Inner portion of main body
10 Main body
12 Internal universal lancet receiving receptacle
12a Aperture of internal universal lancet receiving receptacle
12b Bottom (terminus) of internal universal lancet receiving receptacle
12c Internal side wall of universal lancet receiving receptacle
14 Internal rib elements of universal lancet receiving receptacle
16 Suction securing element
18 Lancet securing means

### 200 Lancet References

20 Lancet
20a Distal end of lancet, comprising needle element N
20b Proximal end of lancet, comprising body or handle portion H
20c Cap

### 300 Anatomical References

30 Hand (30r for right hand)
31 Thumb (first digit)
32 Index finger (second digit)
33 Middle finger (third digit)
34 Ring finger (fourth digit)
35 Little finger (fifth digit)
36 Wrist
37 Palm of hand

### 400 Orientation References (with Respect to the Hand, Wrist and Forearm)

42 Distal (towards the tips or ends of the digits of the hand)
43 Proximal (towards the wrist and forearm)
44 Dorsal (the back of the hand)
45 Palmar (the palm of the hand)
46 Lateral (the side portion of the hand opposite the thumb)
48 Forearm pronation (palm of the hand facing down)
49 Forearm supination (palm of the hand facing up)

It should be noted that the invention is directed for use with a lancet 20 and that the various embodiments of the invention are not limited to any particular design, form or type of lancet 20, and further, that no particular design, form or type of lancet 20 is a claimed feature or limitation of the invention.

The within description and illustrations of various embodiments of the invention are neither intended nor should be construed as being representative of the full extent and scope of the present invention. While particular embodiments of the invention are illustrated and described, singly and in combination, it will be apparent that various modifications and combinations of the invention detailed in the text and drawings can be made without departing from the spirit and scope of the invention. For example, references to materials of construction, methods of construction, specific dimensions, shapes, utilities or applications are also not intended to be limiting in any manner and other materials and dimensions could be substituted and remain within the spirit and scope of the invention. Accordingly, it is not intended that the invention be limited in any fashion. Rather, particular, detailed and exemplary embodiments are presented.

The images in the drawings are simplified for illustrative purposes and are not necessarily depicted to scale, although effort has been made to do so where feasible. To facilitate understanding, identical reference numerals are used, where possible, to designate substantially identical elements that are common to the figures, except that suffixes may be added, when appropriate, to differentiate such elements.

Although the invention herein has been described with reference to particular illustrative and exemplary physical embodiments thereof, as well as a methodology thereof, it is to be understood that the disclosed embodiments are merely illustrative of the principles and applications of the present invention. Therefore, numerous modifications may be made to the illustrative embodiments and other arrangements may be devised without departing from the spirit and scope of the present invention. It has been contemplated that features or steps of one embodiment may be incorporated in other embodiments of the invention without further recitation.

### DETAILED DESCRIPTION

A more detailed description of the invention now follows.

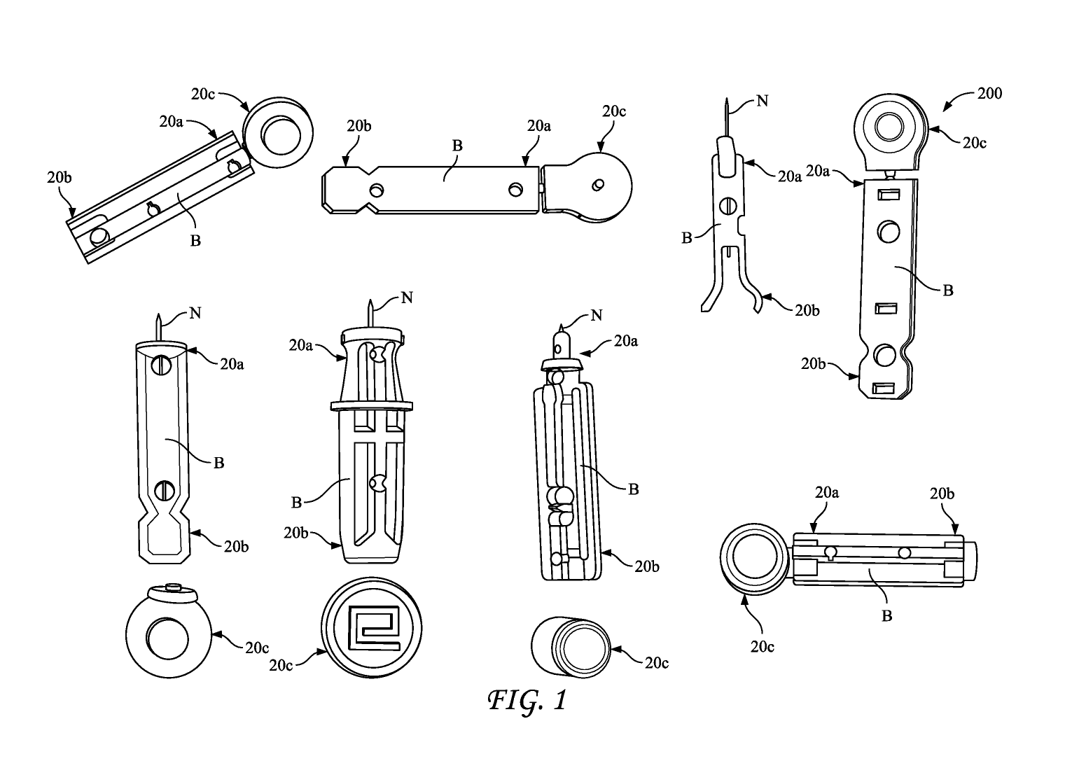

FIG. 1 is perspective side view of eight (8) types or variations of commercially available blood lancets 20. Each lancet 20 is generally comprised of a distal end 20a comprising a sharp, pointed needle element N and a proximal end 20b comprising a handle or body portion B which may be manually held by a user or secured within a lancet device or lancet holding device. Lancets are often sold in bulk packages, are generally disposable and disposed in a safe, secure manner after use, and are typically sold with a protective cap portion 20c that covers the needle element N. At the time of use, the user removes the cap portion 20c, thereby exposing the sharp needle element N. Some of the lancets 20 of FIG. 1 are depicted with their respective caps 20c removed, thereby exposing the needle element N, while other lancets are depicted with the cap 20c covering the needle element N.

Lancet needles N generally comprise various widths known as gauges. Lancet gauges generally range from about 20 to about 34 and the higher the gauge, the smaller the perforation the lancet makes. For example, a 23-gauge lancet creates a larger puncture wound in the skin than does a 30-gauge lancet. In addition, the needle elements N may also comprise various lengths, as certain users may require a longer lancet needle N length to perforate thicker skin.

The needle portion N of the lancet 20 is generally comprised of surgical steel or other suitable metal alloy commonly used for such purposes. The wider handle or body portion B is usually comprised of an injection molded thermoplastic, for example Polyamide (Nylon), ABS, Acetal, Polycarbonate (PC), Polyethylene (PE), Polypropylene (PP), or Polyvinyl chloride (PVC), which may further be blended or glass reinforced, though any other suitable material, such as a lightweight plastic or metal that is capable of being machined. Alternatively, the lancet body portion B may be comprised of the same surgical steel comprising the blade/needle element N or any other appropriate metal alloy as is generally known in the art. In addition, as depicted in FIG. 1, the handle or body portion B of the lancet 20 may define one or more holes, features and other elements that aid in coupling the lancet 20 to a specific, corresponding lancet device or lancet holding device, particularly proprietary commercial devices specifically designed for accepting, securing and using a particular lancet design.

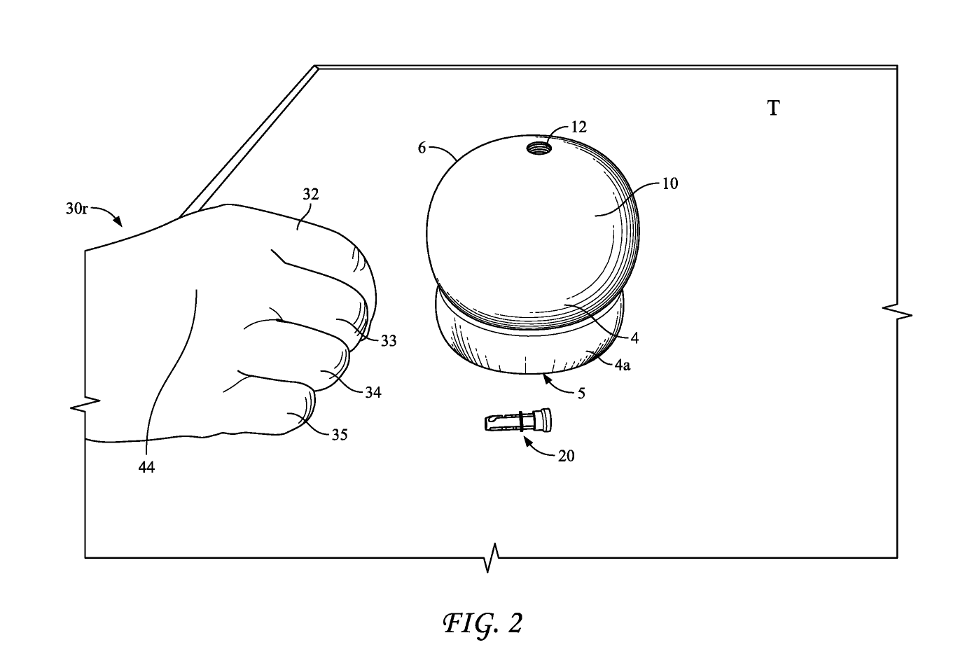

FIG. 2 is perspective view of an embodiment of the invention resting on a flat stable surface, such as a table T. Further depicted in FIG. 2 is a hand 30 of a user of the invention and a lancet 20, with cap 20c attached, laying on the flat surface immediately adjacent to the embodiment.

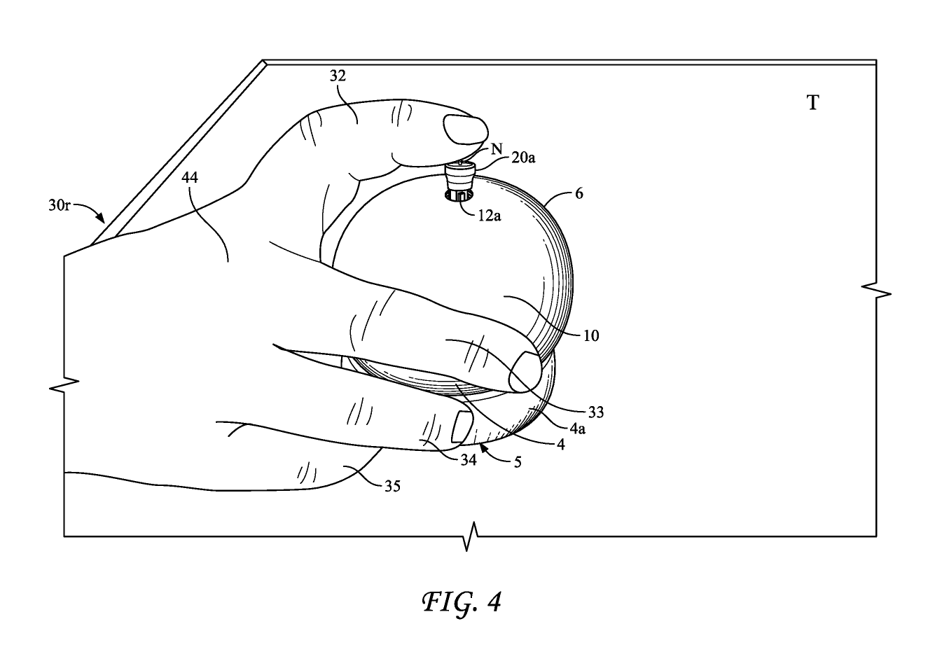

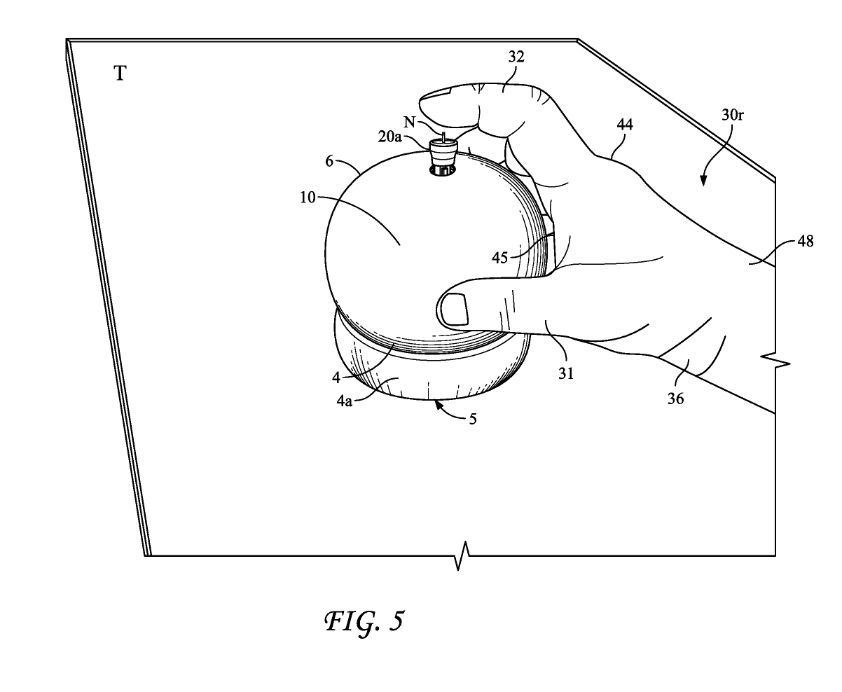

The embodiment of FIG. 2 is comprised of a main body portion 10, in this case, defined by an outer contoured surface 6 that is substantially convex about an inner portion 8 (see FIG. 6 for reference to inner portion 8) and the three-dimensional shape of which is substantially spherical or ellipsoidal in design and nature. The outer contour 6 of the main body portion 10 is ergonomically optimized in shape and size to accommodate an easy grasp of the user's hand 30, as further described below, without undue stretching of the digits of the hand or in such manner to cause pain or discomfort. That is, the size, shape and dimensions of the main body portion 10, as defined by its outer contoured surface 6, is of any ergonomically directed size, shape and dimension that (referring to FIGS. 4-5) may be readily received by and accommodated within a palmar 45 surface a hand 30, including the palm 37 and the palmar 45 surface of the digits thereof, for easy grasping between the thumb (first digit) 31 and opposing digits middle finger (third digit), ring finger (fourth digit) and if necessary the little finger (fifth digit) when the forearm is pronate 48 and the wrist 36 of the hand is resting on the flat surface.

The embodiment of FIG. 2 is further comprised of a universal lancet receiving receptacle 12—in this embodiment, located on the top outer surface 6 and extending into the inner portion 8 in a substantially vertical fashion—for detachably securing (holding) a lancet 20 for use by the individual to obtain a blood sample. The universal lancet receiving receptacle 12 is generally defined as comprising a cavity space within the inner portion 8 of the main body 10 and (referring to FIG. 6, et al) is comprised of an aperture 12a, substantially circular in shape and defined by a first diameter on the outer contour surface of the main body portion 10, an internal terminus portion defined by a bottom 12b (thereby defining the length of the internal cavity of the receptacle 12 into the inner portion 8) also substantially circular in shape and defined by a second diameter, and an inner side wall portion 12c that extends from the aperture 12a to the bottom portion 12b in a substantially cylindrical in shape, but wherein the first diameter of the aperture 12a is greater than the second diameter of the internal terminus portion or bottom 12b, as detailed further below. The embodiment of FIG. 2 is further comprised of a bottom portion 4, which as depicted, further features a base element 4a having flat bottom surface 5 for stable placement of the embodiment on a flat surface, such as, for example, a table T.

FIG. 2 further depicts a user's hand, in this case a right hand 30r, in a pronate 48 anatomical orientation, with the wrist 36 (not shown) resting on the table T, dorsal 44 side of the hand facing up. Also depicted are the little finger (fifth digit) 35, ring finger (fourth digit) 34, middle finger (third digit) 33 and index finger (second digit) 32.

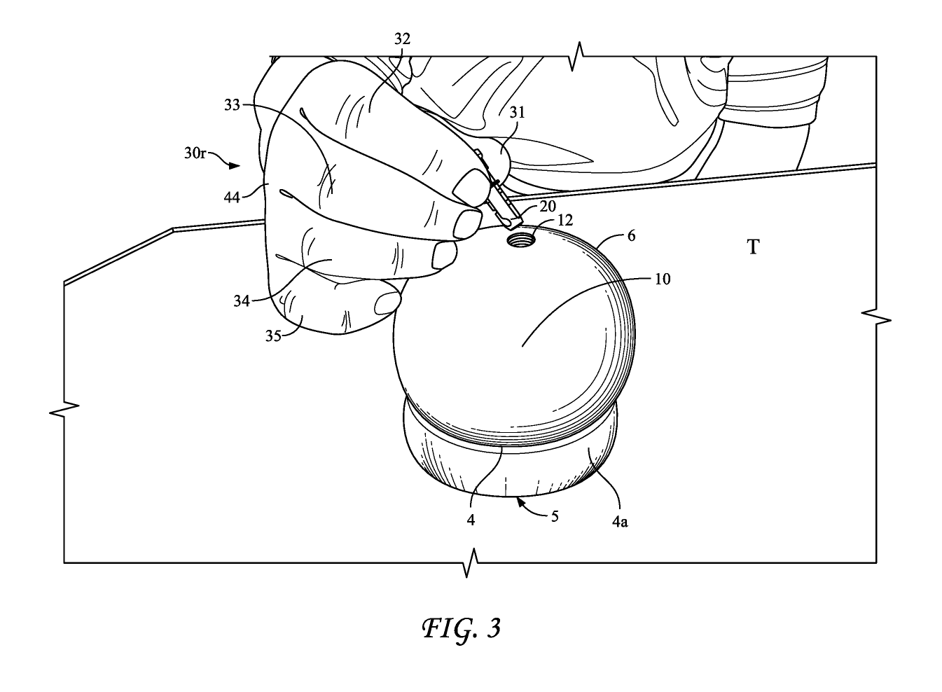

FIG. 3 is a similar perspective view of the embodiment of the invention of FIG. 2 and further depicting a user placing a lancet 20 in an embodiment with a right hand 30r. As noted, the invention is directed towards an easy-to-use, ergonomic lancet holding device that meets the needs of individuals with limited or no operational function or use in one arm or hand, i.e., allows for simple, easy single-handed operation. The user of FIG. 3 can readily insert and detachably secure and hold a lancet 20 within the universal lancet receiving receptacle 12 of the embodiment only using the operative right hand 30r. The lancet 20 is depicted being held by the user between the thumb (first digit) 31 and index finger (second digit) 32 of the right hand 30r.

FIG. 4 is a similar perspective view of the embodiment of the invention of FIGS. 2-3, demonstrating further use of the invention. In FIG. 4, the lancet 20 is detachably secure in the universal lancet receiving receptacle 12. The body portion B of the lancet 20 is firmly held within the receptacle 12, thereby presenting a secure and stable lancet needle element N to the user for puncturing the skin of a digit of the right hand 30r. As shown in FIG. 4, a the body portion B of the lancet 20 is substantially detachably secured within the receptacle 12, which may vary depending on the size, shape and type of lancet 20 used by the individual. The distal end of the lancet 20b protrudes from the aperture 12a of the receiving receptacle 12 in a substantially vertical fashion relative to the flat surface or table upon which the embodiment rests. The cap 20c of the lancet 20 has been removed by the user and the lancet needle element N is fully exposed and presented for use.

Continuing with FIG. 4, the user is ready to use the invention to puncture the skin of the distal portion of the index finger 32 of the right hand 30r and obtain a lancet-induced blood sample. This is performed by the user by tapping the distal portion of the index finger 32 on the needle element N. The outer contour 6 of the main body 10 is ergonomically shaped to readily accommodate the palmar surface of the hand and digits, thereby providing the user with easy-to-grasp functionality. Because the user may readily and easily grasp the outer contoured surface 6 of the main body 10 of the embodiment—without excess stretching or discomfort of the hand, digits, wrist or forearm—the user is better able to cognitively focus upon and direct the requisite muscular precision to obtain a quick blood sample. The ergonomic shape of the invention, as expressed in the outer contour of the main body portion 6, specifically steadies the hand and forearm and allows for precision guidance of the appropriate digit, in this case the index finger 32 of the right hand 30r, over the lancet needle N.

Continuing with FIG. 4, stability is further provided by and through the flat stable surface, in this case a table T, upon which the embodiment rests. The flat bottom surface 5 rests cleanly and securely on the table. As such, the user can place his or her arm on the table as well and need not focus upon or direct energy attempting to hold up the operative right arm and right hand 30r to obtain a blood sample. Instead, the full weight of the arm rests on the table T, thereby providing a more stable and secure environment for lancing the skin. Moreover, the user is able to accomplish the entire task all with one functioning or operative arm and hand.

Continuing further with FIG. 4, the user grasps the ergonomic outer contour surface 6 of the main body 10 of the embodiment between the thumb 31 on one side of the body 10 (not visible in FIG. 4) and the remaining opposing digits on the other side of the body 10. In the example depicted in FIG. 4, the middle finger (third digit) 33 and ring finger (fourth digit) 34 provide the primary opposing grasping force and the fifth digit (little or pinky finger) 35 primary acts to stabilize the hand and rest upon the table. The right hand 30r is presented in forearm pronation orientation 48 and the wrist 36 rests on the table. The dorsal 44 portion of the hand (back of the hand) faces up. This leaves the second digit (index finder) 32 freely available and provides optimum stability for puncturing the distal palmar portion of that finger. The foregoing merely provides an exemplary use of the embodiment and is not intended in any way to limit the use thereof. While different users may find more optimal means of utilizing the invention to obtain a blood sample, the disclosed method of use of the embodiment presents substantial stability and is ideally suited for individuals with limited or no use of one arm or hand and individuals that suffer from tremors or shaking.

FIG. 5 is the similar perspective view of the embodiment (and use thereof) as in FIG. 4 from an opposite angle. In this view, the thumb (first digit) 31 is now visible. As is depicted in FIG. 5, the thumb 31 is only slightly grasping the main body 10. Because of the unique outer contour surface 6 of the main body 10, the amount of muscle force or strength required from a user to securely grasp the device is distributed among several of the digits, thereby equalizing exertion. In addition, the flat bottom surface 5 of the embodiment maintains stability on the flat surface, thereby making it less likely to be moved inadvertently. As depicted in the drawings, the invention is optimally used on a flat surface, particularly a table T, so that the user may rest his or her one working arm and hand on the table. The sum of these features and conditions translates into an effective, stable and easy-to-use lancet holding device.

FIG. 6 is a cross section view of a portion of an embodiment of the invention, in particular, a cross section view of a lancet receiving receptacle 12 of an embodiment. In particular, FIG. 6 depicts a cross sectional planar view of a portion of the main body 10 of an embodiment of the invention wherein said cross section plane intersects a center radius R of the universal lancet receiving receptacle 12. Depicted immediately above the aperture 12a of the receiving receptacle 12 is a lancet 20, with a distal end 20a and a proximal end 20b. Comprising the distal end 20a is the needle element N. The proximal end 20b of the body B of the lancet 20 is ready for insertion by a user into the aperture 12a of receiving receptacle 12, as demonstrated by the wide arrow pointing from the proximal end 20b towards the aperture 12a.

The embodiment depicted in FIG. 6 further comprises an outer contour surface 6 of the main body 10 and an inner portion 8 of the main body 10. The inner portion 8 is depicted in diagonal lines. Extending into the inner portion 8 is the receiving receptacle 12 of sufficient size and dimension for receiving and detachably securing the lancet 20. The receiving receptacle 12 is defined by an internal cavity in the inner portion 8, comprised of an aperture 12a on the outer surface 6, said aperture 12a being substantially circular in shape and defined by an approximate first diameter. In an embodiment, the internal terminus or bottom 12b of the cavity of the receiving receptacle 12 is also substantially circular in shape and defined by an approximate second diameter. The receiving receptacle 12 is further defined by an internal side wall portion 12c which comprises various internal diameters of the receptacle 12 that are defined according to the approximate first diameter of the aperture 12a and the approximate second diameter 12b of the internal terminus of the receptacle 12 and wherein the internal side wall portion 12c extends from the aperture 12a on one end thereof to the internal terminus 12b on the other end thereof. The internal receptacle 12 is substantially cylindrical in shape.

In the embodiment of FIG. 6, the approximate first diameter of aperture 12a is greater than the approximate second diameter of the internal terminus or bottom portion 12b. Protruding from the internal side wall portion 12c of the receptacle 12 and extending inwards towards the center radius of the receptacle are a plurality of gripping means, in the disclosed embodiment internal rib elements 14, which are depicted in FIG. 6. as hashed lines. As the lancet 20 is inserted into the receptacle 12 by a user, the proximal end 20b thereof comes into contact with the gripping means 62, in this case, internal rib elements 14, which detachably secure the lancet 20. As the lancet 20 is further inserted into the receptacle 12 of the disclosed embodiment of the figure, given the increasing reduction in the diameter of the receptacle, as defined by the shape of its internal side wall portion 12c according to the first diameter of the aperture 12a and the second diameter of the internal terminus 12b, and given the material comprising the gripping means, the body portion B of the lancet comes into greater contact (and thus, resistance) with the internal rib elements 14 (gripping means), which further tighten the hold on the lancet 20, thereby providing greater stability of the lancet 20 for use by the user. Depending on the proprietary design of a lancet 20, the proximal end 20b thereof may be inserted the entire length of the receiving receptacle 12 and come into contact with the internal terminus 12b thereof. Other lancet designs may not allow for full insertion into receiving receptacle 12. Regardless, the gripping means, in the case of FIG. 6, internal rib elements, provide a universal secure means of holding the lancet 20.

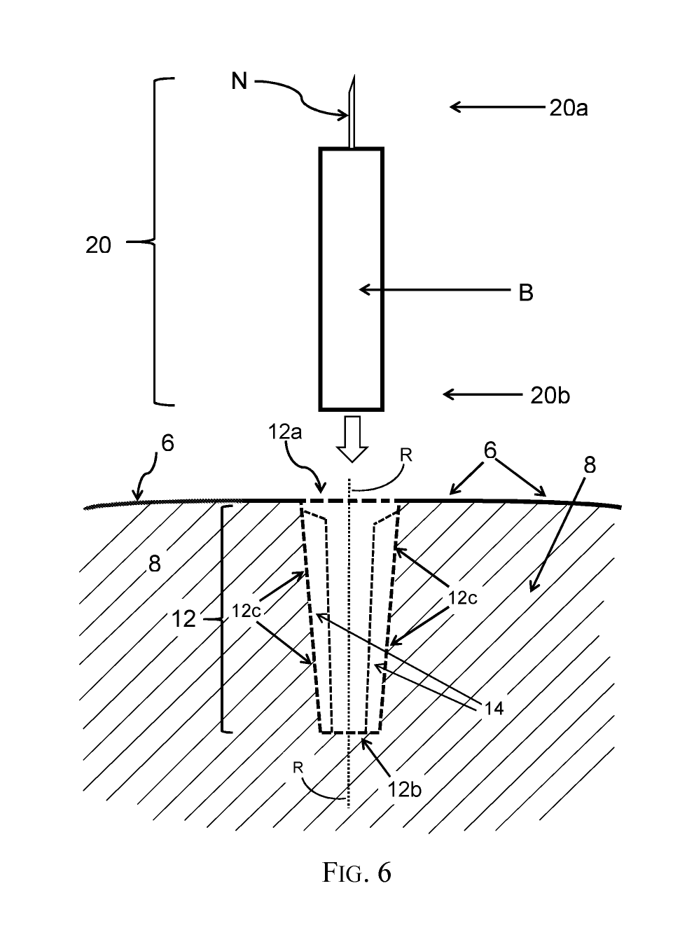

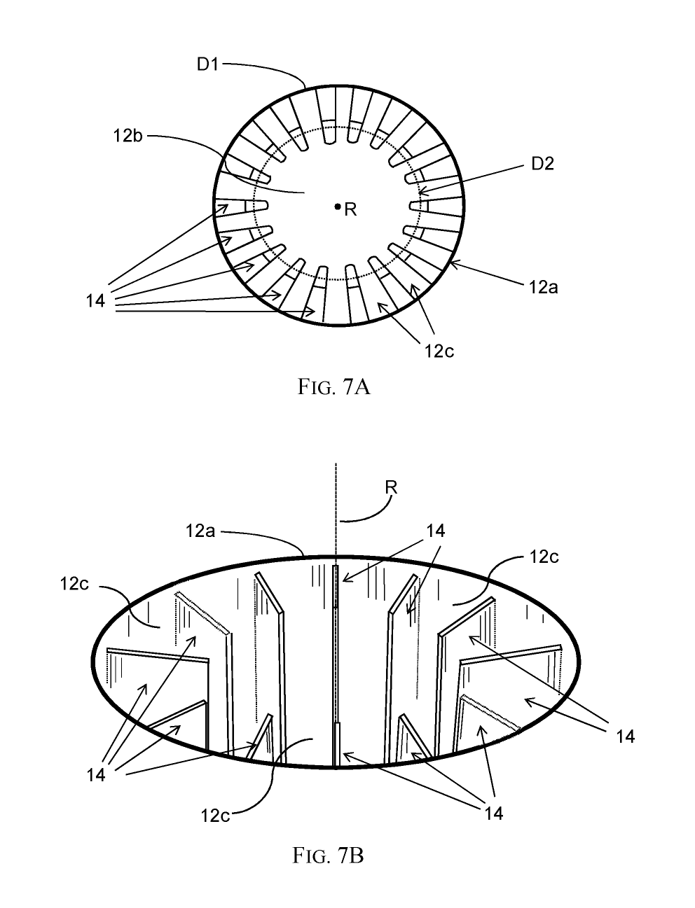

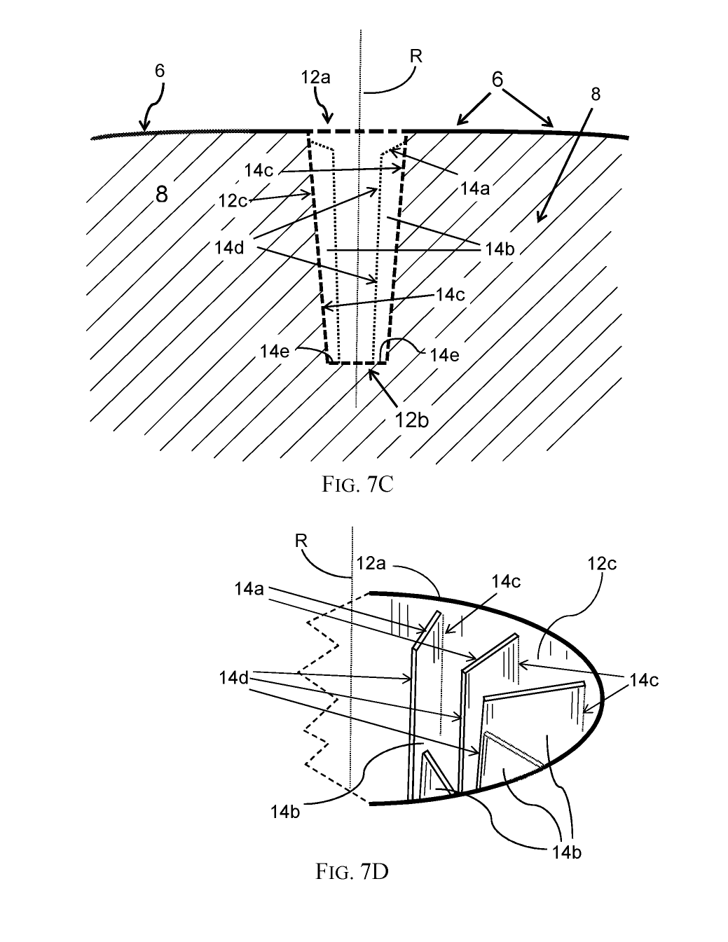

FIG. 7A is top view of a portion of an embodiment of the invention. In particular, FIG. 7A is a top view of a receiving receptacle 12 of an embodiment depicting a plurality of the internal rib elements 14, which serve as a gripping means for a lancet 20. The aperture 12a to the receptacle is substantially circular in shape and defined by a first diameter D1 around a center radius point R. A plurality of internal rib elements 14 comprising a gripping means of the receiving receptacle 12 extend inward from the internal side wall 12c of the receiving receptacle into the internal cavity defined therein. The internal terminus of the receptacle or bottom 12b is also substantially circular in shape and is defined by a second diameter D2 around a center radius point R. In the embodiment of FIG. 7A the second diameter D2 is less than the first diameter D1, and as a result, the substantially circular shape of the internal terminus or bottom 12b comprises less radial area than the substantially circular shape of the aperture 12a and the perimeter thereof. The plurality of internal ribs 14 depicted in FIG. 7A extend from approximately the aperture 12a to the internal terminus 12b of the receptacle.

FIG. 7B is a perspective view of a portion of an embodiment of the invention. In particular, FIG. 7B is a perspective view of a receiving receptacle 12 of an embodiment similar to that of FIG. 6 and FIG. 7A depicting a plurality of the internal rib elements 14, which serve as a gripping means for a lancet 20. The aperture 12a to the receptacle is substantially circular in shape and defined by a first diameter D1 (reference number not shown) around a center radius point R. A plurality of internal rib elements 14 comprising a gripping means of the receiving receptacle 12 extend inward from the internal side 12c of the receiving receptacle into the internal cavity defined therein. In the embodiment of FIG. 7B the plurality of internal ribs 14 extend approximately the length of the receiving receptacle 12, from approximately the aperture 12a to the internal terminus 12b of the receptacle. The internal gripping ribs 14 are comprised of a first upper end 14a, two side portions 14b, a proximal end 14c attached to the internal side 12c of the receiving receptacle 12, a distal end 14d that is defined by the inner, gripping edge of the gripping ribs and a second lower end 14e (not depicted). See also FIGS. 7C-7D for further detail.

The gripping rib elements 14 depicted in the various embodiments of the drawings are not necessarily to scale and illustrative of exemplary embodiments. As shown in FIG. 7A, the gripping ribs 14 are defined by a wider proximal base 14c than the same elements depicted in the embodiments of FIGS. 7B and 7D. The gripping rib elements 14 of the receptacle may be comprised of any shape and dimension necessary to provide a gripping means and detachably secure (hold) a lancet 12 in the receptacle for use by an individual.

FIG. 7C is a cross section view of a portion of an embodiment of the invention as that depicted in FIG. 6. Specifically, FIG. 7C depicts a cross sectional planar view of a portion of the main body 10 of an embodiment of the invention wherein said cross section plane intersects a center radius of the universal lancet receiving receptacle 12. The various features of the gripping elements 14 are depicted in the drawing: a first upper end 14a, side portions 14b, a proximal end or base 14c attached to the internal side 12c of the receiving receptacle 12, a distal end 14d that is defined by the interior, gripping edge of the gripping ribs 14 and a second lower end 14e that is congruous with and attaches to the internal terminus or bottom 12b of the receptacle.

FIG. 7D is a perspective view of a portion of an embodiment of the invention as that depicted in FIG. 7B. FIG. 7D is a perspective view of a receiving receptacle 12 of an embodiment similar to that of FIG. 6 and FIG. 7A-7B depicting a plurality of the internal rib elements 14, which serve as a gripping means for a lancet 20. The various features of the gripping elements 14 are depicted in detail in the drawing of FIG. 7D: a first upper end 14a, side portions 14b, a proximal end or base 14c attached to the internal side 12c of the receiving receptacle 12, a distal end 14d that is defined by the interior, gripping edge of the gripping ribs 14. Not depicted is a second lower end 14e that is congruous with the internal terminus or bottom 12b of the receptacle.

The internal rib elements 14 may extend from about the aperture 12a to the terminus 12b of the receptacle 12. Some of the plurality of rib elements 12a may be of less length. The length of the proximal end or base 14c need not be the same for each rib element 14. In addition, the length between the proximal base end 14c of the rib element and its internal distal end 14d may be of the same amount for all rib elements in an embodiment or said length may vary among the plurality of rib elements in an embodiment. The rib elements 14, or any gripping means used, may be of any size, shape or dimension to accommodate the variety of lancets available for detachable securement to the device for use.

In the embodiment of FIGS. 6, 7A-7D, the gripping means are comprised of a plurality of flexible rib elements 14. The gripping means of the invention is specifically not limited to the depicted flexible rib elements 14 and is intended to encompass other means suitable for such specific use. For example, in an embodiment, the gripping means may be comprised of a plurality of flexible protrusions or fingers extending from the internal side 12c of the receiving receptacle 12 into the interior cavity defined therein. The flexible fingers would function substantially as the flexible rib elements 14—as the lancet 20 is inserted into the receptacle 12, the body portion B thereof would encounter increased resistance in proportion to the amount that the lancet 20 has been inserted into the receptacle 12. The resistance created by a plurality of flexible, yet sturdy, finger protrusions would thereby snuggly hold the lancet 20 in a stable, firm manner for use by the individual. The invention may utilize any number of commonly known means for simple, detachable securement (holding) of small implements. The plurality of flexible internal rib elements and the plurality of flexible internal finger protrusions are examples.

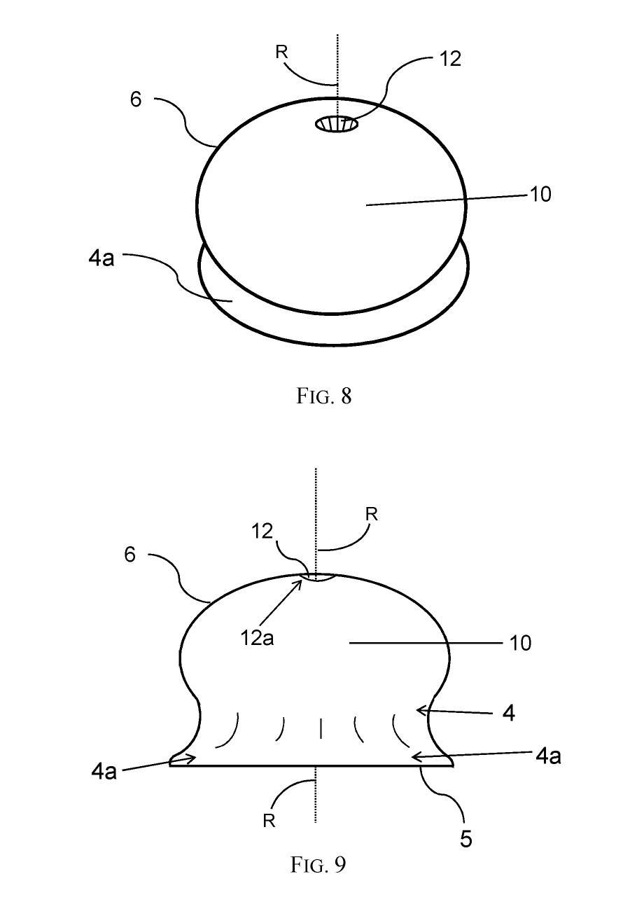

FIG. 8 is a simple perspective view of an embodiment of the invention depicting the ergonomic contour shape 6 of the main body portion 8 to easily accept and accommodate the palmar surface of a hand seeking to gently grasp the device for use.

FIG. 9 is a simple side view of an embodiment of the invention depicting the ergonomic contour shaped outer surface 6 of the main body 8 to easily accept and accommodate the palmar surface of a hand seeking to gently grasp the device for use. The embodiment of FIG. 9 comprises a uni or single body design, wherein the main body portion 10 is comprised of a congruous, continuous bottom portion 4, including the base element 4a and bottom surface 5. In the embodiment, the convex outer contour 6 shape of the main body portion 10 flairs out at the bottom portion 4 to form a base element 4a that is wider than the bottom portion 4 of the main body 10, thereby forming a concave contour between the main body 10 and the base element 4a. The shape of the embodiment of FIG. 9 provides additional ergonomic function and greater stability to the device for use on a surface. Alternatively, the base element 4a may be a separate piece that is securely attached to a correspondingly shaped surface of the bottom portion 4 of the main body 10, such as by glue, acrylics, plastic, adhesives or other securing means that are generally well known, including various hardware means.

Embodiments of the invention depicted in FIGS. 2-9 are substantially symmetrical in shape and design and are therefore suited for use by either the right or left hand. That is, the outer contour shape 6 of the main body 10 is of a size, design and shape that the device may be readily received and grasped by either the right or left hand; the embodiments are symmetrical and therefore of design, size and shape to accommodate the palmar surface of the either hand with the thumb (first digit) and opposing fingers (digits) easily grasping the device without any strain.

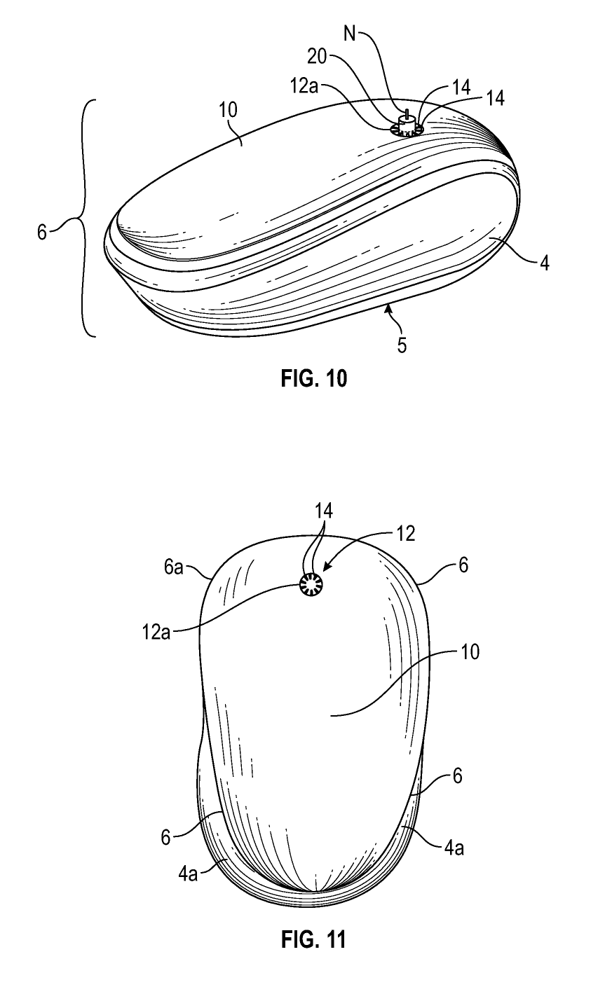

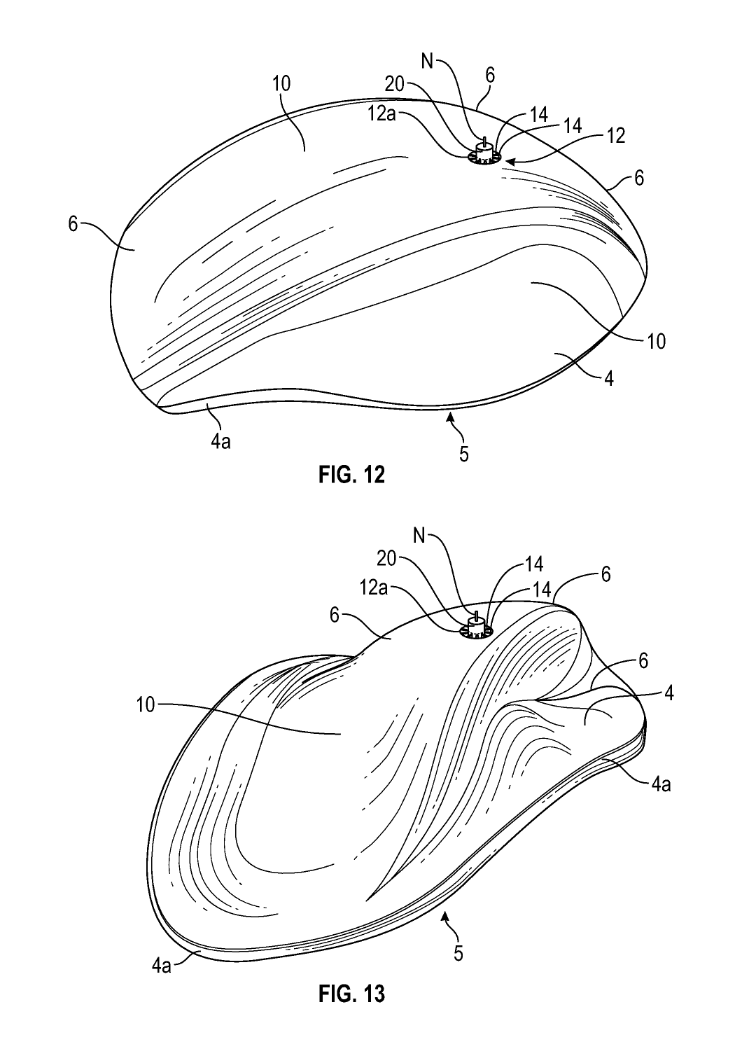

FIGS. 10-13 provide additional embodiments of the invention wherein the outer contour shape surface 6 of the main body portion 10 are asymmetrical in overall design and shape and provide unique ergonomic function to a specific hand of a user, i.e., a first embodiment may be designed for use the right hand and a mirror image isomer of said first embodiment (now a second embodiment) designed for use by the left hand. Various users may find further stability to the arm or hand in using embodiments that are specifically directed towards the right hand or the left hand and not a universal design, such as that depicted in prior embodiments, that may be used by either the right or the left hand. The embodiments of FIGS. 10-11 provide specific right and left hand ergonomic function and stability as may be required by a specific individual.

The invention is not limited to the designs of the embodiments depicted in the drawings and the nature and scope of the invention is specifically directed towards any suitable size, shape and design that is directed to accommodate the anatomy of the human hand for easy receipt thereof by the palmar surface and easy grasping by the digits of the hand. Various embodiments may come in a variety of sizes for different size hands, e.g., extra-small, small, medium, large, extra-large, etc.

Embodiments of the invention may be comprised of various materials suited for such uses. Plastics, resins, composites are particularly suited. Suitable common plastics may include, but are not limited to, Polyethylene terephthalate (PET), Polyethylene (PE), High-density polyethylene (HDPE), Polyvinyl chloride (PVC), Low-density polyethylene (LDPE), Polypropylene (PP), Acrylonitrile butadiene styrene (ABS), Polycarbonate (PC), Polycarbonate/Acrylonitrile Butadiene Styrene (PC/ABS). Various specialty plastics and thermoplastics may include, but are not limited to, Maleimide/bismaleimide, Melamine formaldehyde (MF), Polyetheretherketone (PEEK), Polyetherimide (PEI), Polyimide, Polymethyl methacrylate (PMMA), Polytetrafluoroethylene (PTFE), Urea-formaldehyde (UF). Such materials and composites may comprise the inner portion 8 and/or the outer contoured surface 6 of various embodiments. Various metals and metal alloys may be used or added to the main body 10 or any other portion of an embodiment to provide additional weight and added stability to the device. The device may also be comprised from wood or any other suitable material.

Embodiments that are comprised, in part, of a metal or metal alloy, or other heavy-weighted material, may provide additional stability because of the added weight. For example, in an embodiment, the inner portion 8 of the base element 4a of the bottom portion 4 is comprised, in whole or in part, of a metal alloy that provides a “paper weight” function to the embodiment, thereby providing additional stability and keeping the device from moving when used by an individual suffering from tremors. The added base weight of the design therefore provides functionality and advances the objectives of the invention in providing a stable lancet holding device for individuals that would benefit from a sturdy, stable and weighted device. In an additional embodiment, the entire inner portion of the device may be comprised of metal, metal alloy or any other heavy-weighted material to provide even additional weight and stability. Such embodiments may weigh as much as 8-16 ounces, or more, depending on the needs of a user.

Various embodiments may comprise an inner portion 8 that is either solid in composition or it may also comprise one or more hollow sections. The embodiments may be produced by any generally known means, such as extrusion or injection molding, casting and the like. The embodiments of the invention may also be constructed from any other suitable material that provides sufficient firmness and stability for the purposes disclosed herein. Some materials may be better suited than others and in embodiments of the invention, the outer contoured surface 6 and the bottom surface 5 are comprised of any of the forgoing or other materials that may withstand cleaning with common household soaps and materials and may be readily cleaned. In an embodiment, the device is water resistant and/or water proof.

In an embodiment, the bottom surface 5 is further comprised of a non-skid surface material to provide further stability to the device. Non-skid materials may be applied to the bottom surface 5 as are commonly known in the art to provide the required non-skid functionality. Such non-skid surfaces may include, but are not be limited to, non-skid rubber coatings and surfaces to prevent the device from moving when in use. Rubber would include natural rubbers and composites, as well as synthetic rubber-like composites that provide non-skid functionality.

The internal flexible ribbed gripping elements (such as, for example, the plurality of flexible internal rib elements 14 or the plurality of flexible internal finger protrusions) may be comprised of rubber, plastics, composites, resins or any other flexible, yet durable, material suitable for such uses. Such materials may include, but not be limited to, Polyacrylate Rubber (ACM), Ethylene-acrylate Rubber (AEM), Polyester Urethane (AU), Bromo Isobutylene Isoprene (BIIR), Polybutadiene (BR), Chloro Isobutylene Isoprene (CIIR), Polychloroprene (CR), Chlorosulphonated Polyethylene (CSM), Epichlorohydrin (ECO), Ethylene Propylene (EP), Ethylene Propylene Diene Monomer (EPDM), Polyether Urethane (EU), Perfluorocarbon Rubber (FFKM), Fluoronated Hydrocarbon (FKM), Fluoro Silicone (FMQ), Fluorocarbon Rubber (FPM), Hydrogenated Nitrile Butadiene (HNBR), Polyisoprene (IR), Isobutylene Isoprene Butyl (IIR), Acrylonitrile Butadiene (NBR), Polyurethane (PU), Styrene Butadiene (SBR), Styrene Ethylene Butylene Styrene Copolymer (SEBS), Polysiloxane (SI), Vinyl Methyl Silicone (VMQ), Acrylonitrile Butadiene Carboxy Monomer (XNBR), Styrene Butadiene Carboxy Monomer (XSBR), Thermoplastic Polyether-ester (YBPO), Styrene Butadiene Block Copolymer (YSBR), Styrene Butadiene Carboxy Block Copolymer (YXSBR). Such materials may also be used for the bottom surface 5 to provide a non-slip, non-skid bottom of the device. The internal flexible ribbed gripping elements (such as, for example, the plurality of flexible internal rib elements 14 or the plurality of flexible internal finger protrusions) may be solid, semi-solid or hollow, depending on size, shape, design and material(s) used in the composition thereof.

In an embodiment, all portions, features and elements of the device are water resistant, water proof and are made of washable materials so that the invention may be washed and cleaned, especially since the device may occasionally encounter blood.

Further embodiments include a manually activated suction feature on the bottom surface 5 for even greater stability by detachably securing the device to a flat, smooth surface, such as a kitchen counter top or glass table. Additional embodiments include an additional lancet securing means, such as a threaded wing bold detachably threaded through a corresponding threaded aperture and channel connected from an outer portion of the main body 10 to the receiving receptacle 12. By tightening the wing bolt in clockwise fashion, the distal end of the bolt enters the receptacle and an inserted lancet may be manually tightened for use. Other such means for tightening a lancet or detachably securing the embodiment to a flat surface are commonly known and may be directed towards such uses with embodiments of the invention.

This description is neither intended nor should it be construed as being representative of the full extent and scope of the present invention.

This disclosure of the various embodiments of the invention, with accompanying drawings, is neither intended nor should it be construed as being representative of the full extent and scope of the present invention. The images in the drawings are simplified for illustrative purposes and are not necessarily depicted to scale. To facilitate understanding, identical reference terms are used, where possible, to designate substantially identical elements that are common to the figures, except that suffixes may be added, when appropriate, to differentiate such elements.

Although the invention herein has been described with reference to particular illustrative embodiments thereof, it is to be understood that these embodiments are merely illustrative of the principles and applications of the present invention. Therefore, numerous modifications may be made to the illustrative embodiments and other arrangements may be devised without departing from the spirit and scope of the present invention. It has been contemplated that features or steps of one embodiment may be incorporated in other embodiments of the invention without further recitation.

1. A lancet holding device, comprising:
a main body portion, comprising an outer contoured surface, an inner portion and a substantially flat bottom portion for placement and use on a flat surface; and
a lancet receiving receptacle for receiving and detachably securing a lancet
wherein the outer contoured surface is ergonomically shaped to accommodate the palmar surface of a hand for grasping and use by the hand to obtain a blood sample from a lancet detachably secured in the lancet receiving receptacle.

2. The lancet holding device of claim 1, wherein the lancet receiving receptacle is comprised of an internal cavity extending from the outer contoured surface into the inner portion, said internal cavity comprising an aperture on the outer contoured surface and an internal terminus in the inner portion and an internal side wall portion extending from the aperture to the internal terminus.

3. The lancet holding device of claim 2, wherein the aperture and the internal terminus are substantially circular in shape.

4. The lancet holding device of claim 3, wherein the substantially circular shape of the aperture is comprised of a first approximate diameter, the substantially circular shape of the internal terminus is defined by a second approximate diameter, and wherein said first approximate diameter is equal to or greater than said second approximate diameter.

5. The lancet holding device of claim 2, wherein the lancet receiving receptacle further comprises a gripping means for detachably securing the lancet.

6. The lancet holding device of claim 5, wherein said gripping means comprises a plurality of internal rib elements attached to and extending from the internal side wall portion into the internal cavity of the receiving receptacle, said rib elements comprising a first length along the internal side wall portion and a second length, said second length comprising the distance of extension of the rib elements from the side wall portion into the internal cavity of the receiving receptacle.

7. The lancet holding device of claim 6, wherein said first length of the internal rib elements extends substantially from about the aperture of the receiving receptacle to the internal terminus of the receiving receptacle.

8. The lancet holding device of claim 6, wherein said second length of the internal rib elements is substantially the same for each rib element.

9. The lancet holding device of claim 6, wherein said second length of the internal rib elements is comprised of a plurality of lengths distributed among the internal rib elements.

10. The lancet holding device of claim 6, wherein said internal rib elements are comprised of semi-rigid, flexible material to allow insertion of the lancet by the user into the receiving receptacle wherein said internal rib elements provide increasing resistance in proportion to the amount of said insertion.
